(19) United States
(12) Patent Application Publication
(10) Pub. No.: US 2009/0234023 A1
(43) Pub. Date: Sep. 17, 2009

(54) SUBSTITUTED HYDROXYACETOPHENONE DERIVATIVES

(75) Inventors: Jose Agustin Quincoces Suarez, Sao Paulo (BR); Klaus Peseke, Elmenhorst/Lichtenhagen (DE); Ernesto Estrada Roger, Santiago de Compostella (ES)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Riemser Arzneimittel AG, Insel riems (DE)

(21) Appl. No.: 11/571,651

(22) PCT Filed: Jul. 6, 2005
(86) PCT No.: PCT/EP2005/007307

§ 371 Date: Oct. 14, 2008

(30) Foreign Application Priority Data
Jul. 6, 2004 (DE) 10 2004 032 711.4

(51) Int. Cl. A61K 31/12; C07C 45/61; C07D 311/22; C07C 49/255; A61P 31/00

(52) U.S. Cl. 514/689; 568/315; 549/402; 568/337

(57) ABSTRACT

The present invention relates to substituted hydroxyacetophenone derivatives having antiproliferative and antimicrobial properties, to pharmaceutical compositions containing them, as well as to a method of preparing them. Moreover, the hydroxyacetophenone derivatives according to the invention can serve as organic intermediates for the preparation of biologically active compounds.

The invention relates to substituted hydroxyacetophenone derivatives having antiproliferative and antimicrobial properties, to drugs containing them as well as to a method of producing them. Further the hydroxyacetophenone derivatives according to the invention can serve as organic intermediate products to produce biologically active compounds.

U.S. Pat. No. 5,449,794 describes the antibacterial, antiviral or immune-stimulating effect of benzopyran derivatives. WO98/29404 and WO01/60359 also describe the usage of benzopyran derivatives.

The present invention provides substituted hydroxyacetophenone derivatives, a method of producing them as well as their use in the production of an antiproliferative and antimicrobial pharmaceutical composition. The hydroxyacetophenone derivatives according to the invention can further be used as organic intermediate products for the production of biologically active compounds.

The disadvantages of the isolation/extraction from propolis with its variable and locally diverse composition are omitted; the compounds according to the invention are also optimised with regard to their activity.

The compounds named herein are known in literature as starting materials or intermediate products.

The production of 1-[2-(3-methylbut-2-enyloxy)phenyl]ethanone from ortho-hydroxyacetophenone in the presence of sodium hydride as a base in dimethylsulfoxide is described in Gazz. Chim. Ital. 1989, 119, 385-388. The electro-chemical deprotection of 1-[2-(3-methylbut-2-enyloxy)phenyl]ethanone to form ortho-hydroxyacetophenone is described in Tetrahedron 2002, 58 (45), 9289-9296, and the titanium-catalysed deprotection is described in Tetrahedron Letters 1999, 40 (46), 8121-8124.

The ytterbium triflate-catalysed deprotection of 1-[4-(3-methylbut-2-enyloxy)phenyl]ethanone to form the respective phenol derivative is described in J. Org. Chem. 1998, 63 (24), 9103-9104. The acid-catalysed synthesis of 1-[4-(3-methylbut-2-enyloxy)phenyl]ethanone starting from 1-[(4-hydroxy)phenyl]ethanone and 2-methyl-3-buten-2-ol is described in Tetrahedron 2003, 59 (27), 5091-5104.

The synthesis of several chalcone derivatives is described in JP 54019948. Amongst others, 1-[2,4-bis(3-methylbut-2-enyloxy)phenyl]ethanone is also used as starting material.

According to the invention, the substituted hydroxyacetophenone derivatives of the general formula III can be produced:

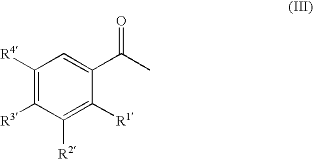

wherein R^{1′}, R^{2′}, R^{3′} and R^{4′} are independently from each other hydrogen, a prenyl group, a 1,1-dimethylallyl group or a 3-methylbut-2-enyloxy group, with the proviso that at least one of R^{1′}, R^{2′}, R^{3′} and R^{4′} is hydrogen and that at least one of R^{1′}, R^{2′}, R^{3′} and R^{4′} is a 3-methylbut-2-enyloxy group, by converting a hydroxyacetophenone derivative of the general formula I:

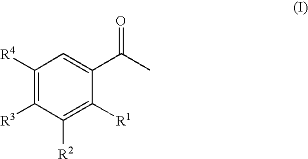

wherein R¹, R², R³ and R⁴ is independently from each other hydrogen, a prenyl group, a 1,1-dimethylallyl group or a hydroxy group, with the proviso that at least one of R¹, R², R³ and R⁴ is hydrogen and that at least one of R¹, R², R³ and R⁴ is a hydroxy group, in the presence of a base, at a reaction temperature of 10 to 50° C., in an organic solvent with a 3-methylbut-2-enylhalide of the formula II:

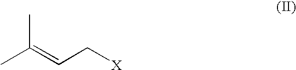

wherein X is chlorine, bromine or iodine, [O-allylation according to the general procedure 1 (GP1), see also the following formula scheme (1)].

Preferably compounds of the general formula III are produced according to GP1, wherein R^{1′} and/or R^{3′} is a 3-methylbut-2-enyloxy group, i.e. they are produced from compounds of the general formula I, wherein R¹ and/or R³ is a hydroxy group.

The O-allylation is preferably conducted in dimethylformamide (DMF) as solvent at a reaction temperature of 20 to 40° C. in the presence of the base potassium carbonate (see GP1).

The O-allylated compounds of the general formula III which are produced this way can then be reacted according to the general procedures 2 or 3 (GP2 or GP3) in N,N-diethylaniline and at a reaction temperature of 160 to 220° C. (preferably under reflux) to form the corresponding C-prenylated or C-alkylated compounds of the general formula IV:

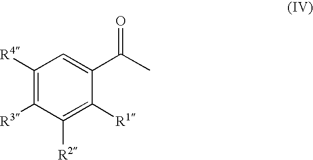

wherein R^{1″}, R^{2″}, R^{3″} and R^{4″} are independently from each other hydrogen, a prenyl group, a 1,1-dimethylallyl group or a hydroxy group, with the proviso that at least one of R^{1″}, R^{2″}, R^{3″} and R^{4″} is a prenyl group or a 1,1-dimethylallyl group and that at least one of R¹″, R^{2″}, R^{3″} and R^{4″} is a hydroxy group, [see the following formula scheme (2)].

Preferably compounds of the general formula IV are produced according to GP2 or GP3, wherein R^{1″} and/or R^{3″} is a hydroxy group.

The compounds of the general formula IV, wherein R^{1″} is a hydroxy group, can further be reacted with at least two mole equivalents of phosphorus oxychloride (POCl₃) in DMF at a reaction temperature of 40 to 50° C. (preferably 45° C.) to form the respective 4-oxo-4H-chromen-3-carbaldehyde derivatives of the general formula V, wherein R^{2″}, R^{3″} and R^{4″} are independently from each other hydrogen, a prenyl group, a 1,1-dimethylallyl group or a hydroxy group, with the proviso that at least one group of R^{2″}, R^{3″} and R^{4″} is a prenyl group or a 1,1-dimethylallyl group, [see the following formula scheme (3)].

Formula scheme 1: O-allylation of phenolic hydroxy groups
with 3-methylbut-2-enylbromide

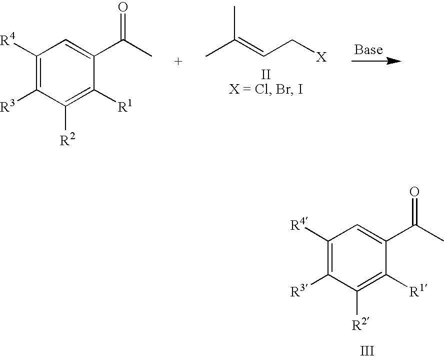

| | |
|---|---|
| educts I | reaction products III |

| | |
|---|---|
| Ia (o-Hydroxyacetophenon^{e)} | IIIa (L1): R^{1′} = 3-methyl-but-2-enyloxy |
| R¹ = OH | R^{2′}, R^{3′}, R^{4 ′} = H |
| R², R³, R⁴ = H | IIIb (LP1): R^{1′}, R^{2′}, R^{4′} = H |
| Ib (p-Hydroxyacetophenon^{e)} | R^{3′} = 3-methyl-but-2-enyloxy |
| R³ = OH | IIIc (2,4O): R^{1′}, R^{3′} = 3-methyl-but-2- |
| R⁴, R³, R¹ = H | enyloxy |
| Ic (2,4-Dihydroxyacetophenone) | R^{2′}, R^{4′} = H |
| R¹, R³ = OH | IIId (L3): R^{1′} = 3-methyl-but-2-enyloxy |
| R², R⁴ = H | R^{4′} = 3-methyl-but-2-enyl |
| Id (L2) | R^{2′}, R^{3′} = H |
| R¹ = OH | IIIe (LMK6): R^{1′} = 3-methyl-but-2- |
| R², R³ = H | enyloxy |
| R⁴ = 3-methyl-but-2-enyl | R^{2′} = 1,1-dimethylallyl |
| Ie (LMK3) | R^{3′}, R^{4′} = H |
| R¹ = OH | IIIf (LP3): R^{1′}, R^{2′} = H |
| R² = 1,1-dimethylallyl | R^{3′} = 3-methyl-but-2-enyloxy |
| R³, R⁴ = H | R^{4′} = 3-methyl-but-2-enyl |
| If (LP2) | reaction |
| R¹, R² = H | |
| R³ = OH | |
| R⁴ = 3-methyl-but-2-enyl | |

Formula scheme 2: Claisen Rearrangement
of the 3-methylbut-2-enyloxyphenyl ethers

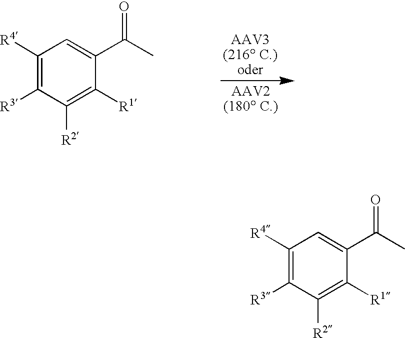

| 3-methyl-but-2-enyloxyphenyl ether III | reaction products IV |
|---|---|
| IIIa (L1): R^{1′} = 3-methyl-but-2-enyloxy | IVa (L2): R^{1″} = OH |
| R^{2′}, R^{3′}, R^{4′} = H | R^{2″}, R^{3″} = H |
| IIIb (LP1): R^{1′}, R^{2′}, R^{4′} = H | R^{4″} = 3-methyl-but-2-enyl |
| R^{3′} = 3-methyl-but-2-enyloxy | IVb (LP2): R^{1″}, R^{2″} = H |
| IIIc (2,4O): R^{1′}, R^{3′} = 3-methyl-but-2- | R^{3″} = OH |
| enyloxy | R^{4″} = 3-methyl-but-2-enyl |
| R^{2′}, R^{4′} = H | IVc: R^{1″}, R^{3″} = OH |
| IIId (L3): R^{1′} = 3-methyl-but-2-enyloxy | R^{2″}, R^{4″} = 3-methyl-but-2-enyl |
| R^{4′} = 3-methyl-but-2-enyl | IVd: R^{1″} = OH |
| R^{2′}, R^{3′} = H | R^{3″} = H |
| IIIe (LMK6) R^{1′} = 3-methyl-but-2- | R^{2″}, R^{4″} = 3-methyl-but-2-enyl |
| enyloxy | IVe: R^{1″} = OH |
| R^{2′} = 1,1-dimethylallyl | R^{2″} =1,1-dimethylallyl |
| R^{3′}, R^{4′} = H | R^{3″} = H |
| IIIf (LP3): R^{4′} = 3-methyl-but-2-enyl | R^{4″} = 3-methyl-but-2-enyl |
| R^{3′} = 3-methyl-but-2-enyloxy | IVf: R^{1″} = H |
| R^{1′}, R^{2′} = H | R^{2″} = 3-methyl-but-2-enyl |
| IIIa (L1): R^{1′} = 3-methyl-but-2-enyloxy | R^{3″} = OH |
| R^{2′}, R^{3′}, R^{4′} = H | R^{4″} = 3-methyl-but-2-enyl |
| | IVg (LMK3): R^{1″} = OH |
| | R^{2″} = 1,1-dimethylallyl |
| | R^{3″}, R^{4″} = H |
| | reaction |

Formula scheme 3: Heterocyclisation of the
phenols according to Vilsmeier-Haack

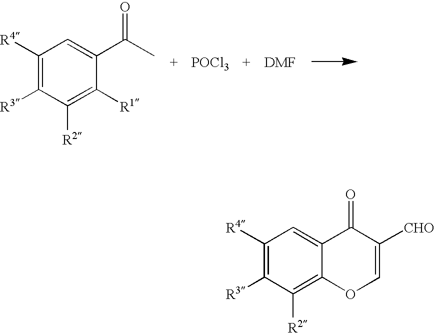

| | 4-oxo-4H-chromen-3- |
|---|---|
| phenols IV | carbaldehydes V |
| IVa (L2): R^{2″}, R^{3″} = H | Va (LMK21): R^{2″}, R^{3″} = H |
| R^{4″} = 3-methyl-but-2-enyl, R^{1″} = OH | R^{4″} = 3-methyl-but-2-enyl |
| IV c: R^{3″} = OH | Vc: R^{2″}, R^{4″} = 3-methyl-but-2-enyl |
| R^{2″}, R^{4″} = 3-methyl-but2-enyl | R^{3″} = OH |
| IV d: R^{2″}, R^{4″} = 3-methyl-but2-enyl | Vd: R^{2″}, R^{4″} = 3-methyl-but-2- |
| R^{3″} = H; R^{1″} = OH | enyl |
| IVe (LMK7): R^{2″} = 1,1-dimethylallyl | R^{3″} = H |
| R^{3″} = H; R^{1″} = OH | Ve: R^{2″} = 1,1-dimethylallyl |
| R^{4″} = 3-methyl-but2-enyl | R^{3″} = H |
| IVg (LMK3): R^{2″} = 1,1-dimethylallyl | R^{4″} = 3-methyl-but-2-enyl |
| R^{3″}, R^{4″} = H | Vg: R^{2″} = 1,1-dimethylallyl |
| R^{1″} = OH | R^{3″}, R^{4″} = H |

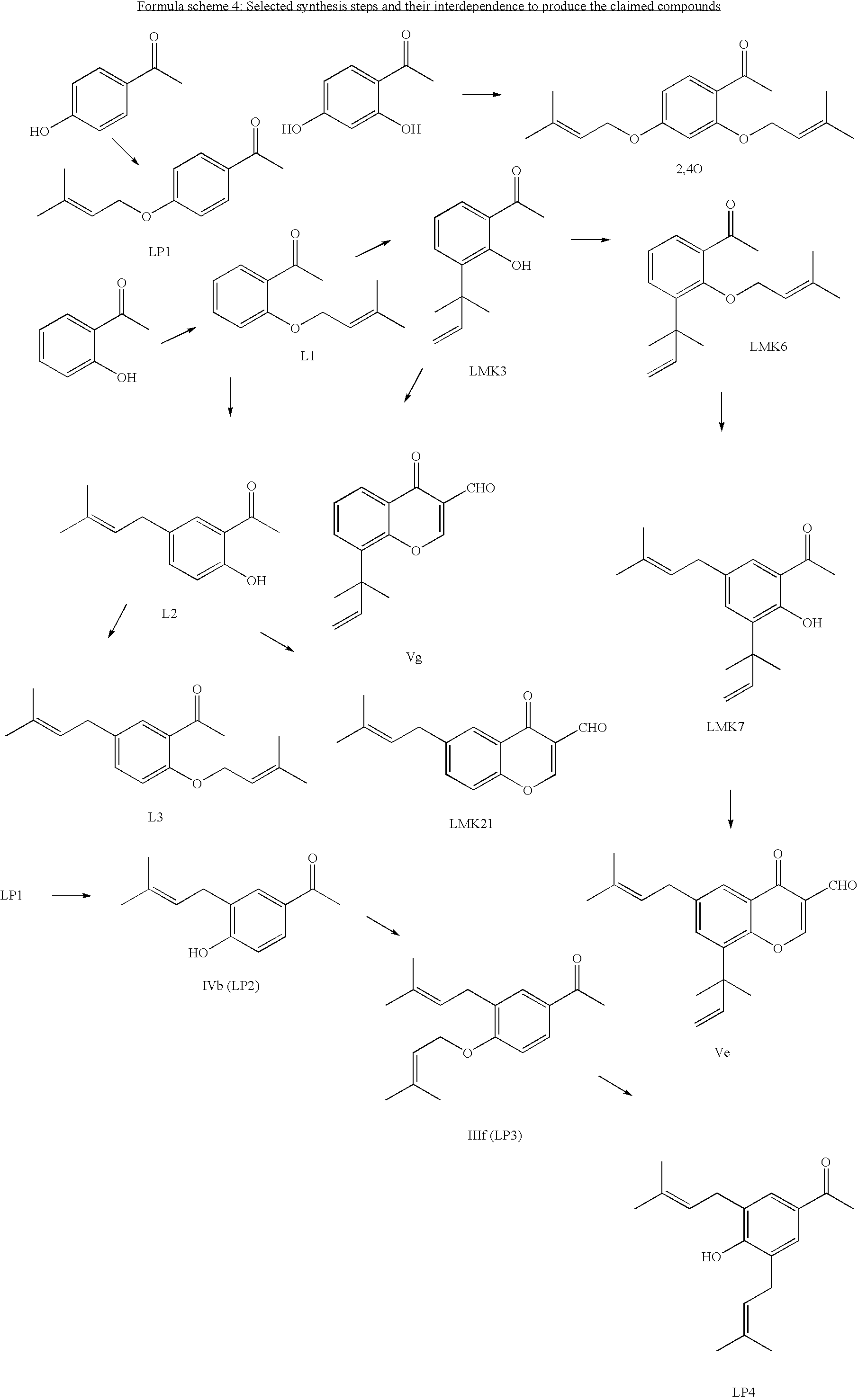

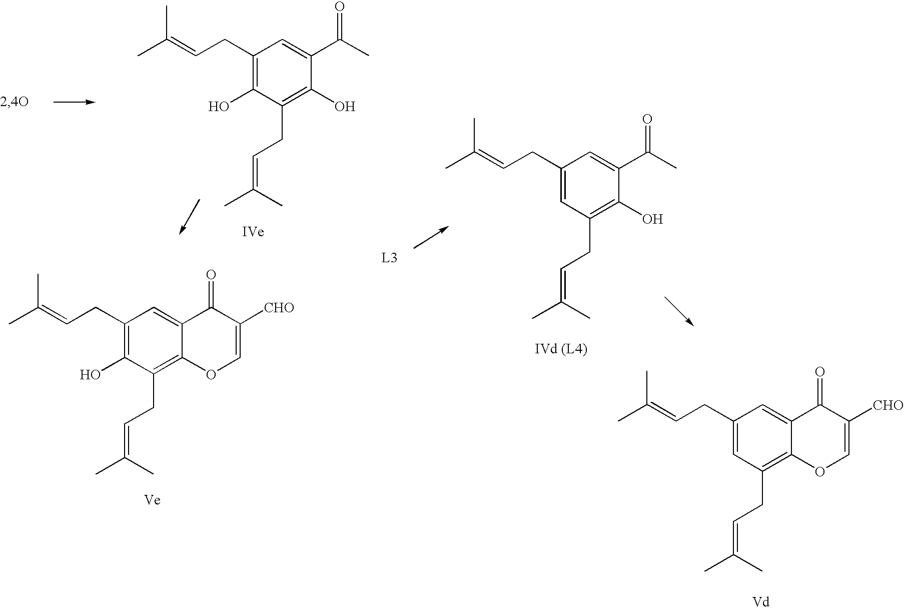

The hydroxyacetophenone derivatives according to formulas III and IV produced according to the aforementioned methods as well as the 4-oxo-4H-chromen-3-carbaldehyde derivatives of formula V exhibit antiproliferative and/or antimicrobial properties and can thus be used according to the invention for the manufacture of antiproliferative or antimicrobial pharmaceutical compositions.

The hydroxyacetophenone derivatives of formulas III and IV as well as the 4-oxo-4H-chromen-3-carbaldehyde derivatives of formula V have not yet been described, except for 1-[2-(3-methylbut-2-enyloxy)phenyl]ethanone, 1-[4-(3-methylbut-2-enyloxy)phenyl]ethanone and 1-[2,4-bis(3-methylbut-2-enyloxy)phenyl]ethanone, and thus they represent another aspect of the present invention.

Preferred compounds of the present invention are the following compounds of formula III:
1-[5-(3-methylbut-2-enyl)-2-(3-methylbut-2-enyloxy)phenyl]ethanone,
1-[3-(1,1-dimethylallyl)-2-(3-methylbut-2-enyloxy)phenyl]ethanone and
1-[5-(3-methylbut-2-enyl)-4-(3-methylbut-2-enyloxy)phenyl]ethanone;
   of formula IV:
1-[2-hydroxy-5-(3-methylbut-2-enyl)phenyl]ethanone,
1-[4-hydroxy-5-(3-methylbut-2-enyl)phenyl]ethanone,
1-[3-(1,1-dimethylallyl)-2-hydroxyphenyl]ethanone,
1-[3-(1,1-dimethylallyl)-2-hydroxy-5-(3-methylbut-2-enyl)phenyl]ethanone,
1-[2-hydroxy-3,5-bis(3-methylbut-2-enyl)phenyl]ethanone,
1-[4-hydroxy-3,5-bis(3-methylbut-2-enyl)phenyl]ethanone and
1-[2,4-dihydroxy-3,5-bis(3-methylbut-2-enyl)phenyl]ethanone; and
   of formula V:
6-(3-methylbut-2-enyl)-4-oxo-4H-chromen-3-carbaldehyde,
6,8-bis(3-methylbut-2-enyl)-7-hydroxy-4-oxo-4H-chromen-3-carbaldehyde,
6,8-bis(3-methylbut-2-enyl)-4-oxo-4H-chromen-3-carbaldehyde,
8-(1,1-dimethylallyl)-6-(3-methylbut-2-enyl)-4-oxo-4H-chromen-3-carbaldehyde and
8-(1,1-dimethylallyl)-4-oxo-4H-chromen-3-carbaldehyde.

### General Procedures (GP) for the Synthesis of Hydroxyacetophenone Derivatives

### GP1: O-allylation of phenolic hydroxy groups with 3-methylbut-2-enyl bromide

6 mmole of potassium carbonate is added to a stirred solution of 2 mmole of the respective hydroxyacetophenone of general formula I in 10 ml dimethylformamide. The solution is stirred at room temperature for 30 minutes under argon atmosphere. Then 3 mmole of 3-methylbut-2-enylbromide are injected into the solution under continuous stirring. After about 8 hours of continuous stirring under inert gas at 40° C., the mixture is poured into 20 ml of an ice/water mixture. The aqueous suspension is extracted three times with 30 ml of each chloroform. The organic phase is then washed with a sodium hydrosulphate solution and then with water. The drying of the chloroform phase is carried out over sodium sulphate. After filtration the solution is concentrated under reduced pressure and the residue is worked up chromatographically.

### GP2: CLAISEN Rearrangement of the 3-methylbut-2-enyloxyphenyl ethers

1 mmole of the respective allyl phenyl ether of general formula III is dissolved in 10 ml N,N-diethylaniline. The solution is heated to 180° C. for 5 hours. After the reaction solution has cooled down, it is taken up in 50 ml chloroform. The chloroform solution is washed three times with hydrochloric acid, 15% and then with water to obtain a neutral pH-value. Thereafter, the chloroform is removed by distillation under reduced pressure and the residue is taken up in 20 ml methanol. A spatula tip of Amberlite IR-120 is added to this solution and the mixture is stirred for 20 minutes. After filtration, the solution is concentrated under reduced pressure and the residue is worked up chromatographically.

### GP3: CLAISEN-COPE Rearrangement of the 3-methylbut-2-enyloxyphenyl ethers

1 mmole of the respective 3-methylbut-2-enyloxyphenyl ether of the general formula III is taken up in 10 ml N,N-diethylaniline. The solution is then heated under reflux for 8 hours (216° C.). After completion of the reaction time, the mixture is dissolved in 50 ml chloroform. The N,N-diethylaniline is extracted with hydrochloric acid, 15% and the remaining organic phase is washed with water until a neutral pH is obtained. The chloroform phase is separated and the chloroform is removed by distillation under reduced pressure. The residue is dissolved in 20 ml methanol and stirred for 20 minutes after having added a spatula tip of Amberlite IR-120. The solution is filtrated and concentrated under reduced pressure. The residue is worked up chromatographically.

### PREPARATION EXAMPLES

### 1. Synthesis of 1-[2-(3-methylbut-2-enyloxy)phenyl]ethanone (L1)

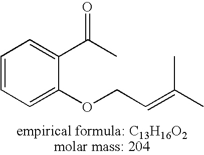

1-(2-Hydroxyphenyl)ethanone is reacted with 3-methylbut-2-enylbromide according to GP1. For the chromatographic work up, a mixture of toluene/ethyl acetate=9/1 was used as eluent.

yield: 85% as colourless liquid; measured Rf-value (TLC, toluene: ethyl acetate 9:1): 0.41

### Structural Identification of the Synthesised Compound L1 by Nuclear Magnetic Resonance (NMR)- and Infrared Spectroscopy (IR):

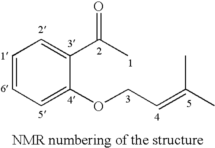

¹H-NMR (DMSO-d₆): 7.6 (d, 2H, H-1′/H-5′), 7.1 (d, 2H, H-2′/H-6′), 5.44 (m, 1H, H-4), 4.6 (d, 2H, H-3), 2.6 (s, 3H, CH₃CO), 1.8 (s, 3H, CH₃ prenyl), 1.7 (s, 3H, CH₃ prenyl).

¹³C-NMR (CDCl₃): 200.1 (CO), 158.3 (C-4′), 138.3 (C-5), 133.5 (C-6′), 130.4 (C-2′), 128.5 (C-3′), 120.4 (C-1′), 119.1 (C-4), 112.7 (C-5′), 65.3 (C-3), 32.0 (C-1), 25.7 (CH₃), 18.2 (CH₃).

IR (recorded as a film): 3072, 3028 (═CH), 2976, (CH₃), 2879 (CH₂), 1674 (C=0), 1236 (C—O) cm⁻¹.

### 2. Synthesis of 1-[2-hydroxy-5-(3-methylbut-2-enyl)phenyl]ethanone

### L2

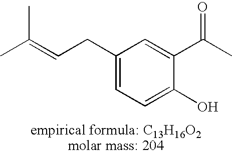

1-[2-(3-Methylbut-2-enyloxy)phenyl]ethanone (L1), obtained in preparation example 1, is reacted according to GP 3. For the work up, a mixture of toluene/ethyl acetate (9.5/0.5) was used as eluent.

yield: 70% as colourless liquid; measured Rf-value (TLC, toluene: ethyl acetate 9:1): 0.79

### Structural Identification of the Synthesised Compound L2 by Nuclear Magnetic Resonance (NMR)-, Infrared (IR)- and Mass Spectrometry (MS):

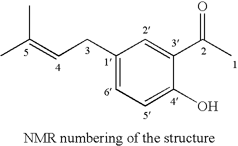

¹H-NMR (DMSO-d₆): 12.1 (s, 1H, OH), 7.5 (d, 1H, H-6′), 7.2 (d, 2H, H-2′/H-5′), 5.3 (m, 1H, H-4), 3.3 (d, 2H, H-3), 2.5 (s, 3H, H-1), 1.8 (s, 3H, CH₃ prenyl), 1.7 (s, 3H, CH₃ prenyl)

¹³C-NMR (CDCl₃): 204.4 (CO), 160.5 (C-4′), 136.8 (C-6′), 133.1 (C-5), 132.1 (C-1′), 129.6 (C-2′), 122.7 (C-4), 119.3 (C-3′), 118.2 (C-5′), 33.3 (C-3), 26.6 (CH₃CO), 25.7 (CH₃ prenyl), 17.8 (CH₃ prenyl)

IR (recorded as a film): 3437 (OH), 3029 (HC=aromatic hydrocarbon), 2971, 2916 (CH₃), 2857, (CH₂), 1642 (C═O), 1589 (C═C) cm⁻¹.

MS (70 eV): 204 M^{+.}; 189 (M^{+.}−15 (CH₃)); 161 (M^{+.}−43 (CH₃CO); 69 (C₅H₉, prenyl); 43 (CH₃Co).

### 3. Synthesis of 1-[3-(1,1-dimethylallyl)-2-hydroxyphenyl]ethanone

### LMK 3

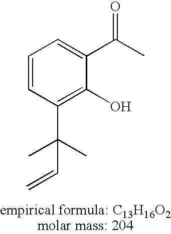

1-[2-(3-Methylbut-2-enyloxy)phenyl]ethanone (L1), obtained in preparation example 1, is reacted according to GP 2. For the work up, a mixture of toluene/ethyl acetate (9.5/0.5) was used as eluent.

yield: 28% as colourless liquid; measured Rf-value (TLC, toluene: ethyl acetate 9.5:0.5): 0.71

### Structural Identification of the Synthesised Compound LMK3 by Nuclear Magnetic Resonance (NMR)-, Infrared (IR)- and Mass Spectroscopy (MS):

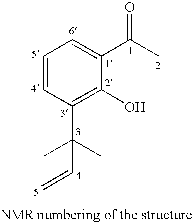

¹H-NMR (CDCl₃): 13.1 (s, 1H, OH); 7.64 (dd, 1H, H-6′); 7.49 (dd, 1H, H-4′); 6.83 (m, 1H, H-5′); 6.25 (dd, 1H, H-4); 5.29 (dd, 1H, vinyl CH₂); 4.97 (dd, 1H, vinyl CH₂); 2.63 (s, 3H, CH₃CO); 1.5 (s, 6H, CH₃-allyl).

¹³C-NMR (75 MHz, CDCl₃): 205.1 (CO); 161.7 (C-2′); 147.2 (C-4, vinyl-CH); 134.1 (C-3′); (C-3′); 129.1 (C-6′), 124.3 (C-1′); 119.6 (C-5′); 110.5 (C-5, vinyl-CH₂); 40.6 (C-3); 27.0 (C-2); 26.8 ((CH₃)2C-5).

IR (recorded as a film): 3435 (OH), 3030 (═CH aromatic hydrocarbon), 2970, 2915 (CH₃), 2855, (CH₂), 1644 (C═O), 1587 (C═C) cm⁻¹.

MS (70 eV): 204 M^{+.}; 189 (M^{+.}−15 (CH₃)); 161 (M^{+.}−43 (CH₃CO); 43 (CH₃CO).

### 4. Synthesis of 1-[3-(1,1-dimethyl-allyl)-2-(3-methyl-but-2-enyloxy)-phenyl]-ethanone

### LMK6

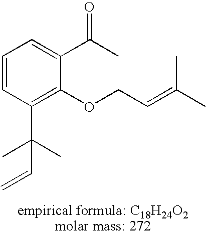

1-[3-(1,1-Dimethylallyl)-2-hydroxyphenyl]ethanone (LMK3), obtained in preparation example 3, is reacted with 3-methylbut-2-enylbromide according to GP1. For the chromatographic work up, a mixture of toluene/ethyl acetate=9.5/0.5 was used as eluent.

yield: 67% as colourless liquid; measured Rf-value (TLC, toluene: ethyl acetate 9:1): 0.37

### Structural Identification of the Synthesised Compound LMK6 by Nuclear Magnetic Resonance (NMR)-, Infrared (IR)- and Mass Spectroscopy (MS):

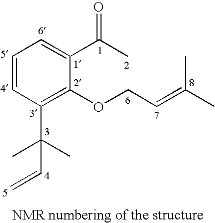

¹H-NMR (CDCl₃): 7.43 (dd, 1H, H-6′); 7.33 (dd, 1H, H-4′); 7.07 (m, 1H, H-5′); 6.21 (dd, 1H, H-4); 5.48 (m, 1H, H-7); 5.03 (dd, 1H, H-5, vinyl CH₂); 4.98 (dd, 1H, H-5, vinyl-CH₂); 4.27 (d, 2H, H-6, O—CH₂); 2.61 (s, 3H, CH₃CO); 1.79 (s, 3H, CH₃-prenyl); 1.63 (s, 3H, CH₃-prenyl); 1.51 (s, 6H, (CH₃)₂C-3).

¹³C-NMR (75 MHz, CDCl₃): 203.0 (CO); 157.0 (C-2′); 148.3 (C-4, vinyl-CH); 136.8 (C-8); 131.3 (C-3′); 128.2 (C-8), 127.6 (C-4′); 123.2 (C-6′); 120.3 (C-1′); 120.1 (C-5′); 120.0 (C-7); 110.3 (C-5); 40.7 (C-3); 29.9 (C-2); 28.2 ((CH₃)₂C-3); 25.7 (CH₃-prenyl); 18.3 (CH₃-prenyl).

IR (recorded as a film): 3032 (═CH aromatic hydrocarbon), 2975, 2917 (CH₃), 2850, (CH₂), 1675 (C═O), 1585 (C═C) cm⁻¹.

MS (70 eV): 272 M^{+.}; 257 (M^{+.}−15 (CH₃)); 229 (M^{+.}−43 (CH₃CO); 43 (CH₃CO).

### 5. Synthesis of 1-[3-(1,1-dimethylallyl)-2-hydroxy-5-(3-methylbut-2-enyl)phenyl]-ethanone

### LMK7

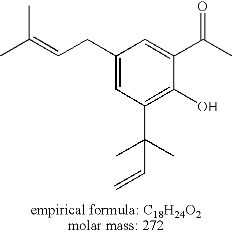

1-[3-(1,1-Dimethylallyl)-2-(3-methylbut-2-enyloxy)phenyl]ethanone (LMK6) obtained in preparation example 4 is reacted according to GP 3. For the chromatographic work up a mixture consisting of toluene/ethyl acetate=9.5/0.5 was used as eluent.

yield: 23% as colourless liquid

### Structural Identification of the Synthesised Compound LMK7 by Nuclear Magnetic Resonance (NMR)-, Infrared (IR)- and Mass Spectroscopy (MS):

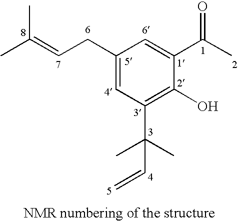

¹H-NMR (CDCl₃): 12.92 (s, 1H, OH); 7.40 (d, 1H, H-6′); 7.31 (d, 1H, H-4′); 6.25 (dd, 1H, H-4); 5.30 (m, 1H, H-7); 5.02 (dd, 1H, H-5, vinyl-CH₂); 4.97 (dd, 1H, H-5, vinyl-CH₂); 3.28 (d, 2H, H-6); 2.61 (s, 3H, CH₃CO); 1.77 (s, 3H, CH₃-prenyl); 1.73 (s, 3H, CH₃-prenyl); 1.50 (s, 6H, (CH₃)₂C-3).

¹³C-NMR (75 MHz, CDCl₃): 204.5 (CO); 160.2 (C-2′); 147.6 (C-4, vinyl-CH); 136.8 (C-8); 134.7 (C-3′); 131.1 (C-4′); 129.9 (C-5′); 123.2 (C-6′); 117.8 (C-1′); 110.1 (C-5); 40.6 (C-3); 38.1 (C-6); 34.1 (CH₃CO); 33.9 ((CH₃)₂C-3); 26.2 (CH₃-prenyl); 18.1 (CH₃-prenyl).

IR (recorded as a film): 3035 (═CH aromatic hydrocarbon), 2973, 2920 (CH₃), 2847, (CH₂), 1643 (C═O), 1580 (C═C) cm⁻¹.

MS (70 eV): 272 M^{+.}; 257 (M^{+.}−15 (CH₃)); 229 (M^{+.}−43 (CH₃CO); 43 (CH₃CO).

### 6. Synthesis of 1-[5-(3-methylbut-2-enyl)-2-(3-methylbut-2-enyloxy)phenyl]-ethanone

### L3

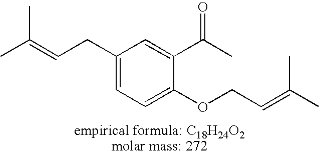

1-[2-Hydroxy-5-(3-methylbut-2-enyl)phenyl]ethanone (L2), obtained in preparation example 2, is reacted with 3-methylbut-2-enylbromide according to GP1. For the chromatographic work up, a mixture consisting of toluene/ethyl acetate=9/1 was used as eluent.

yield: 80%

### Structural Identification of the Synthesised Compound L3 by Nuclear Magnetic Resonance (NMR)- and Infrared Spectroscopy (IR):

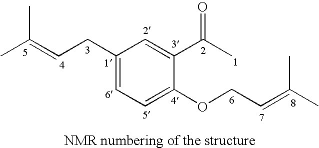

¹H-NMR (DMSO-d₆): 7.5 (d, 1H, H-2′), 7.2 (d, 1H, H-6′), 6.8 (d, 1H, H-5′), 5.5 (m, 1H, H-4), 5.2 (m, 1H, H-7), 4.6 (d, 2H, H-6), 3.2 (d, 2H, H-3), 2.5 (s, 3H, H-1), 1.8 (s, 6H, CH₃ prenyl), 1.7 (s, 6H, CH₃ prenyl).

IR (recorded as a film): 3070, 3026 (═CH), 2970 (CH₃), 2879 (CH₂), 1678 (C═O), 1236 (C—O) cm⁻¹.

### 7. Synthesis of 1-[4-(3-methylbut-2-enyloxy)phenyl]ethanone

### LP1

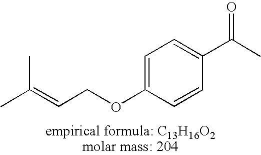

1-(4-Hydroxyphenyl)ethanone is reacted with 3-methylbut-2-enylbromide according to GP1. 1-[4-(3-Methylbut-2-enyloxy)phenyl]ethanone (LP1), precipitated as a solid, is washed with water for the work up and dried at room temperature.

yield: 80% as colourless compound; mp 47° C.

### Quantitative Identification of the Synthesised Compound LP1 by Elementary Analysis:

Calculated mass relations according to formula composition: 76.47% C, 7.84% H

Found mass relations confirm the empirical formula: 76.43% C, 7.81% H

### Structural Identification of the Synthesised Compound LP1 by Nuclear Magnetic Resonance (NMR)-, Infrared (IR)- and Mass Spectroscopy (MS):

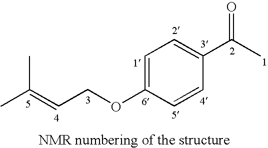

¹H-NMR (DMSO-d₆): 7.9 (d, 2H, H-2′/H-4′), 6.9 (d, 2H, H-1′/H-5′), 5.5 (m, 1H, H-4), 4.6 (d, 2H, H-3), 2.6 (s, 3H, CH₃CO), 1.8 (s, 3H, CH₃ prenyl), 1.7 (s, 3H, CH₃ prenyl)

¹³C-NMR (CDCl₃): 196.7 (CO), 162. 7 (C-6′), 138.8 (C-5), 130.5 (C-2′;C-4′), 130.1 (C-3′), 118.8 (C-4), 114.2 (C-5′; C-1′), 64. 9 (C-3), 26. 2 (C-1), 25.7 (CH₃), 18.1 (CH₃).

IR (recorded as a film): 3072, 3029 (═CH), 2970, 2935 (CH₃), 2875 (CH₂), 1668 (C=0), 1242 (C—O) cm⁻¹.

MS (70 eV): 204 M+, 136 (M+−68), 69 (prenyl).

### 8. Synthesis of 1-[2,4-bis(3-methylbut-2-enyloxy)phenyl]ethanone

### 2,4O

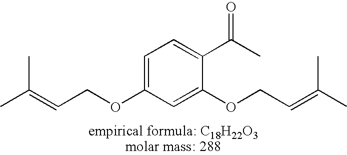

1-(2,4-Dihydroxyphenyl)ethanone is reacted with 3-methylbut-2-enylbromide according to GP1. For the chromatographic work up, a mixture consisting of toluene/ethyl acetate=9/1 was used as eluent.

### Structural Identification of the Synthesised Compound 2,4O by Nuclear Magnetic Resonance (NMR)-Spectrometry:

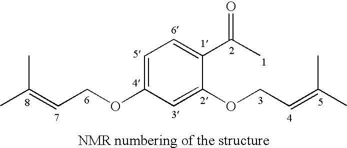

¹H-NMR (DMSO-d₆): 7.6 (d, 1H, H-6′), 6.5 (d, 1H, H-5′), 6.4 (d, 1H, H-3′), 5.6 (m, 1H, H-7), 5.5 (m, 1H, H-4), 4.7 (d, 2H, H-6), 4.6 (d, 2H, H-3), 2.5 (s, 3H, H-1), 1.8 (s, 6H, CH₃ prenyl), 1.7 (s, 6H, CH₃ prenyl).

### 9. Synthesis of 6-(3-methylbut-2-enyl)-4-oxo-4H-chromen-3-carbaldehyde

### LMK21

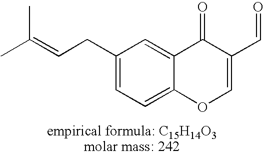

4 mmole of 1-[2-hydroxy-5-(3-methylbut-2-enyl)-phenyl]ethanone (L2) obtained in preparation example 2 is dissolved in 2 ml anhydrous dimethylformamide. 1.29 g phosphorus oxychloride is added dropwise under cooling. The reaction mixture is heated for 1 hour at 45° C. under stirring. The reaction mixture is then poured on ice-water and is then stirred at room temperature for 4 hours. The aqueous phase is extracted with chloroform three times, dried over sodium sulphate, concentrated under vacuum and purified by column chromatography (n-hexane/ethyl acetate=1/0.1)

yield: 11%

### Structural Identification of the Synthesised Compound LMK21 by Nuclear Magnetic Resonance (NMR)-, Infrared (IR)- and Mass Spectroscopy (MS):

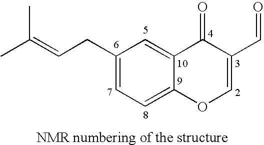

¹H-NMR (CDCl₃): 10.35 (s, 1H, CHO); 8.52 (s, 1H, H-2); 8.07 (d, 1H, H-5); 7.56 (m, 1H, H-7); 7.44 (d, 1H, H-8); 5.32 (m, 1H, ═CH vinyl); 3.46 (d, 2H, CH₂); 1.77 (s, 3H, CH₃-prenyl); 1.73 (s, 3H, CH₃-prenyl).

¹³C-NMR (75 MHz CDCl₃): 188.6 (CHO); 175.3 (C-4); 164.1 (C-2); 156.1 (C-9); 135.7 (C-7), 127.2 (C(CH₃)₂), 125.8 (C-6); 125.1 (C-10); 123.6 (C-5); 119.2 (C-8); 117.9 (═CH-prenyl); 36.5 (CH₂-prenyl); 25.1 (CH₃-prenyl); 18.9 (CH₃-prenyl).

IR (recorded as a film): 3040, 3020 (═C—H), 2975 (CH₃), 2870 (CH₂), 1695 (CHO) e 1645 (CO) cm⁻¹.

MS (70 eV): 242 M^{+.}, 241 (M^{+.}−1(H), 69 (prenyl), 29 (CHO).

### Antitumoral Effects of the Compounds According to the Invention

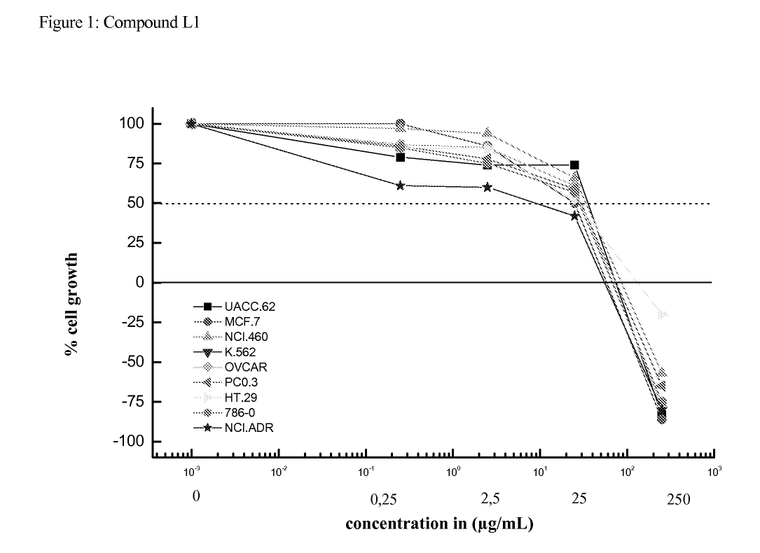

### (Test Results Concerning the Antitumoral Activity of the Synthesised Compounds (See FIGS. 1-6))

The tests were carried out with the following human cancer cell lines:

Lung NCI-460, mamma (breast) normal MCF-7, mamma NCI-ADR (expresses MDR phenotype), skin cancer (melanoma) UACC-62, leukaemia K-562, colon HT-29, renal cancer 786-0, ovarian cancer OVCAR-3 and prostate cancer PC-3 (a gift of the National Cancer Institute, Frederick, Mass., USA).

The cells were cultivated in 25 ml cell culture flasks (Nunc) with 5 ml RPMI 1640 (Gibco BRL, Life Technologies) with 5% of foetal bovine serum.

The adhering cells were trypsinised (0.5 ml trypsin, Nuticell Nutrientes Cellulares). The trypsin was then inactivated by adding 5 ml 5% serum in RPMI 1640. A single cell suspension was prepared by pipetting carefully, the cells were counted, dilutions with an appropriate cell density, varying according to the cell line, were prepared and 100 μl/well were seeded in 96-well microtitre plates. The microtitre plates were pre-incubated for 24 hours at 37° C. to stabilise the cultures. The cells were incubated for 48 hours at 37° C. and 5% CO₂ with 100 μl of the dilutions of the test substances (0.25; 2.5; 25 and 250 μg/ml, in three-fold). Doxirubicin (Sigma Chemical Co.) and tamoxifen (Sigma Chemical Co.) served as positive controls.

The sulforhodamine B (SRB) test for the antiproliferative activity was carried out according to the specifications of Skehan et al. (Journal of National Cancer Institute Vol. 82, pp. 1107-1118, 1990). The cells were fixed for one hour at 4° C. by protein precipitation with 50% trichloroacetic acid (TCA, Sigma Chemicals Co.) (50 μl/well, final concentration 10%)). The supernatant was discarded and the plates were washed with tap water five times. The cells were dyed for 30 minutes with 0.4% SRB in 1% acetic acid (50 μl/well) and subsequently washed four times with 1% acetic acid in order to remove the non-bound dye. The plates were dried and the bound dye was solubilised with 150 μl/well 10 mM Trizma buffer (Sigma Chemicals Co.). The optical density was determined with an automated plate reader (Molecular Devices Max Microplate Reader) at 540 nm. All determinations were conducted as threefold determination. The optical density was calculated with the Excel® program (Microsoft Office Package).

The compounds according to the invention both exhibit cytostatic and cytocidal activity at concentrations ranging between 0.5 to 250 μg/ml. (1-[2-Hydroxy-5-(3-methylbut-2-enyl)ethanone exhibited selective properties without damaging the fibroblasts and was still active at a dilution of 1:2000.

The concentration dependent inhibition of cell growth of chosen compounds according to the invention is shown in FIGS. 1 to 6.

FIG. 1

The compound L1 exhibited cytostatic activity (inhibition of cell growth) against all tested cell lines and cytocidal activity against NCI-460, UACC-62 and NCI-ADR.

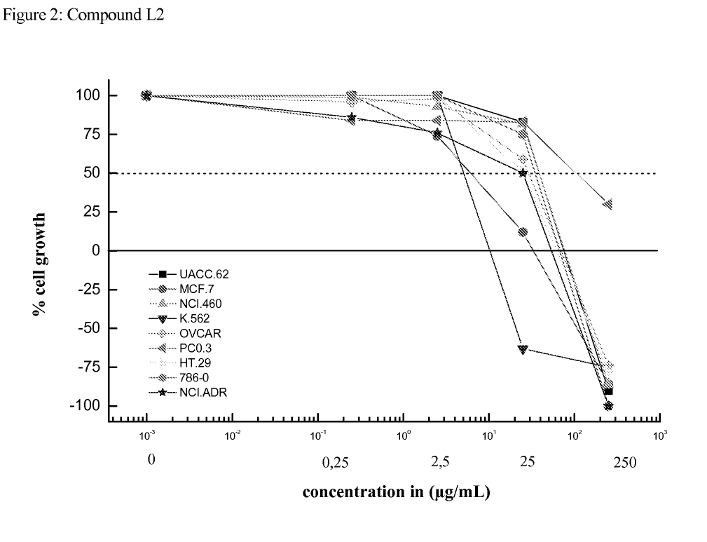

FIG. 2

The compound L2 showed a very strong activity on all cell lines at concentrations ranging between 0.5 and 250 μg/ml.

Moreover, this substance was tested on the following cell lines:

MIAPaCa2 pancreas; C205 colon and T47D breast and for comparison it was tested on the non-pathogenic line: WI38 (fibroblasts).

Compound L2 was selectively active against all 3 tumour lines (i.e. not active against fibroblasts) and was still active at a dilution of 1:2000.

TABLE 1
ED₅₀ (effective dosage which killed 50% of
the human cancer cells, values in μg/ml)

| Compound | NCI460 | UACC62 | MCF7 | NCIADR |
|---|---|---|---|---|
| L1 | 25.0 | 45.0 | 25.0 | 8.0 |
| L2 | 9.0 | 4.2 | 6.0 | 6.0 |

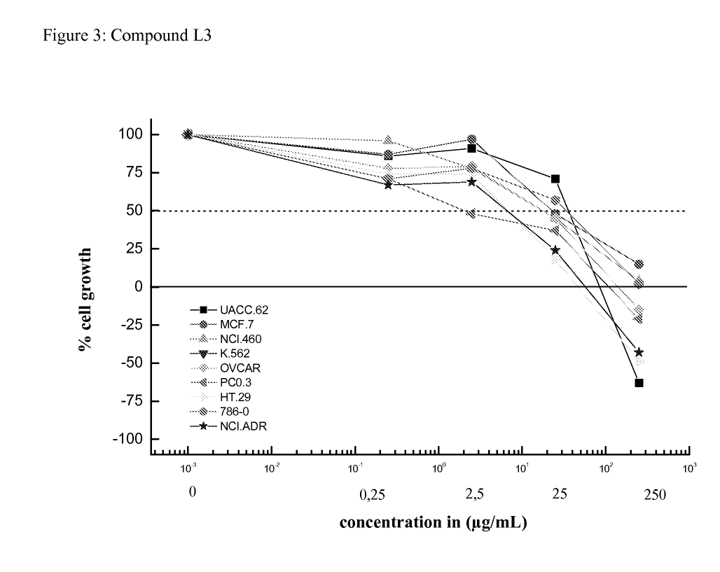

FIG. 3

The cytostatic and cytocidal activity against the tested cell lines as well as the selectivity of compound L3 is illustrated in FIG. 3.

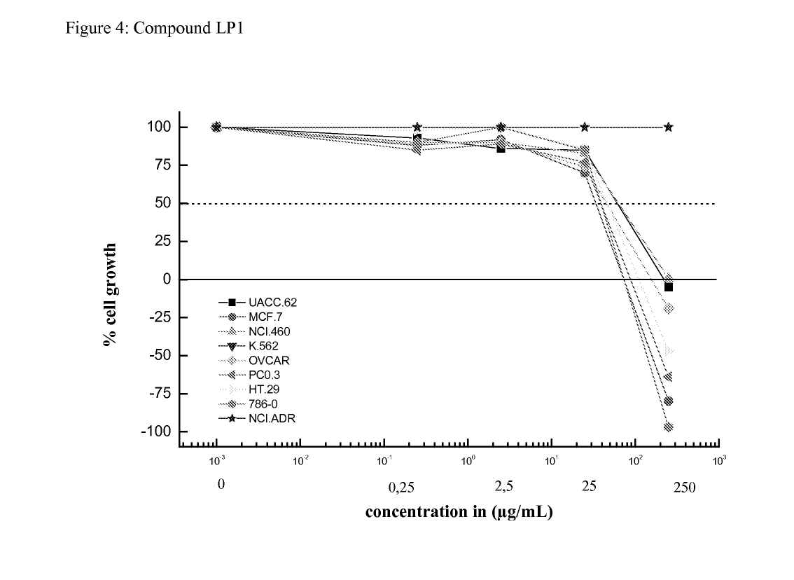

FIG. 4

Compound LP1 showed medium cytostatic activity against all tested cell lines beginning at 25 μg/ml and cytocidal activity beginning at 250 μg/ml.

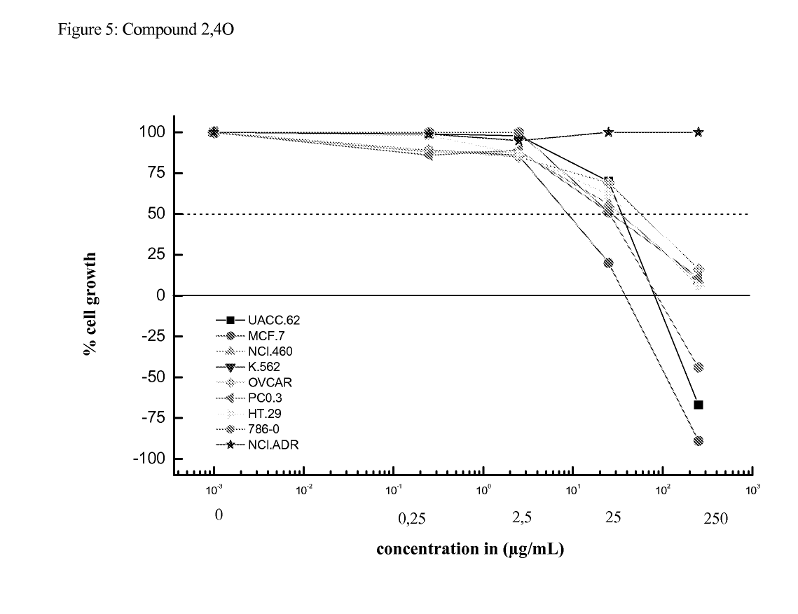

FIG. 5

Compound 2,4O showed medium cytostatic activity against all tested cell lines beginning at 25 μg/ml and cytocidal activity beginning at 250 μg/ml.

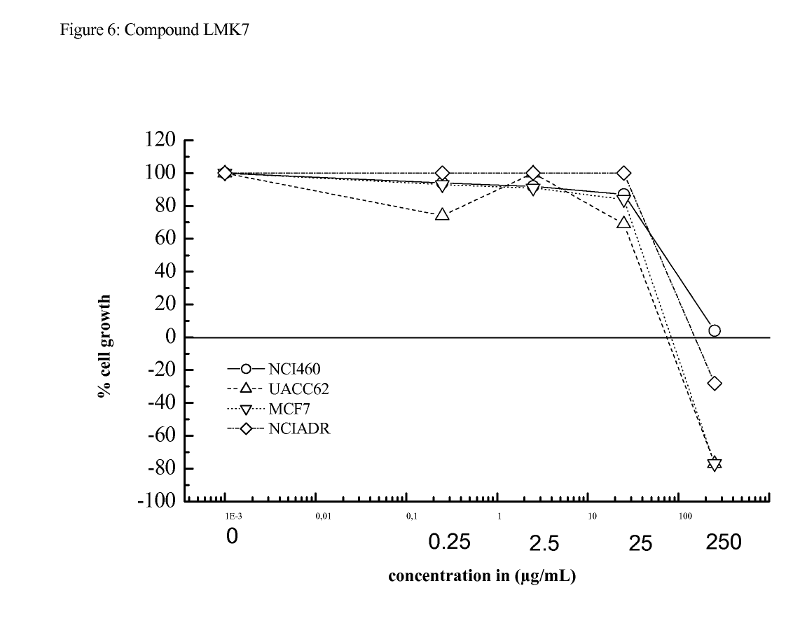

FIG. 6

Compound LMK7 showed cytostatic activity against all tested cell lines beginning at 25 μg/ml, it did not, however, exhibit any cytocidal activity against the tested lung cancer cell line.

### Antimicrobial Tests (Minimal Inhibition Concentration, MIC)

The inoculum for the tests was prepared by diluting the microorganisms in 0.85% NaCl, the dilution was set to level 0.5 according to McFarland and checked at 580 nm in the spectrophotometer. The cell suspensions were diluted to 10⁴ UFC/ml for the application in the tests. The MIC tests were carried out according to the instructions of J. N. P. Eloff (Planta Medica Vol. 64, pp. 711-713, 1998)) in microtitre plates with 96 wells. Serial dilutions of the concentrations of 1.00-0.0015 mg/ml were prepared. Chloramphenicol or nystatin, respectively (Merck) served as control substances in a concentration of 0.062-0.005 mg/ml or 0.0125-0.001 mg/ml, respectively. The inoculum (100 μl) was placed in all wells and the plates were incubated for 48 h at a temperature of 36° C. The antimicrobial activity was detected by adding 20 μl of a 0.5% aqueous solution of TTC (tetrazolium chloride, Merck) per well. The minimal inhibition concentration (MIC) was defined as the lowest concentration of the compound that inhibited visible growth (indicated by TTC stain).

The results of the antimicrobial tests are shown in table 2

TABLE 2
MIC values (in mg/ml)

| | | | | Comparative substance |
|---|---|---|---|---|
| microorganisms | L1 | L2 | L3 | Chloramphenicol |
| B. subitilis | 0.5-1.0 | 0.5-1.0 | >1.0 | 0.02 |
| Micrococcus luteus | 0.25-0.5 | 0.5-1.0 | 0.5-1.0 | 0.05 |
| Rhodococcus equi | >1.0 | >1.0 | >1.0 | 0.04 |
| E. falcium | 0.25 | 0.5-1.0 | 0.5-1.0 | 0.12 |
| Salmonella | 0.25 | 0.5-1.0 | >1.5 | 0.06 |
| choterasuis | | | | |
| Escherichia coli | >1.0 | 0.05 | >1.0 | 0.04 |
| Candida albicans | 0.06 | 0.05 | 0.9 | 0.05** |
| Staphylococcus | 0.5 | >1.0 | >1.0 | 0.02 |
| aureus | | | | |

**Comparative substance against Candida albicans nystatin

TABLE 3
MIC values in (mg/ml)

| | | Comparative substance |
|---|---|---|
| microorganisms | 2,4O | Chloramphenicol |
| S. epidermides | 0.5-1.0 | 0.04 |
| E. coli | 0.5-1.0 | 0.04 |
| Rhodococcus equi | 0.5-1.0 | 0.04 |
| S. falcium | 0.5-1.0 | 0.12 |
| B. subtilis | 0.5-1.0 | 0.02 |
| Micrococcus luteus | 0.5-1.0 | 0.05 |
| E. faecium | 0.5-1.0 | 0.12 |
| Salmonella | 0.5-1.0 | 0.06 |
| S. aureus | 0.5-1.0 | 0.02 |
| Candida albicans | 0.5-1.0 | 0.12** |

**Comparative substance against Candida albicans nystatin

TABLE 4
MIC values in (mg/ml)

| | | Comparative substance |
|---|---|---|
| microorganisms | LP1 | Chloramphenicol |
| S. epidermides | 0.06-0.12 | 0.04 |
| E. coli | >1.5 | 0.04 |
| Rhodococcus equi | >1.5 | 0.04 |
| Candida albicans | >1.5 | 0.12** |
| B. subtilis | >1.5 | 0.02 |
| Micrococcus luteus | >1.5 | 0.05 |
| E. faecium | >1.5 | 0.12 |
| Salmonella | 0.12-0.25 | 0.06 |
| S. aureus | >1.5 | 0.02 |
| S. falcium | >1.5 | 0.12 |

**Comparative substance against Candida albicans nystatin

The above test results demonstrate the excellent antimicrobial (against bacteria and fungi) antiviral or antiproliferative activity of the compounds according to the invention and can therefore be used for therapy and prophylaxis of malignant tumours in human, as for example solid tumours such as colon carcinoma, rectum carcinoma, stomach cancer, thyroid cancer, tongue cancer, bladder cancer, chorion carcinoma, liver cancer, uterine cancer, prostate carcinoma, pharyngeal carcinoma, lung cancer, mamma carcinoma, malignant melanoma, Karposi's sarcoma, brain tumours, neuroblastoma, ovarian carcinoma, testicular cancer, osteosarcoma, pancreas cancer, kidney cancer, hypemephroma, and angioendothelioma; and hematopoietic malignant tumours such as leukaemia or lymphoma.

Furthermore, the compounds according to the invention can be used as drugs against bacteria and mycoses as for example those caused by Candida albicans in case of thrush, pharyngitis, interdigital mycoses and for the treatment of local and systemic mycoses.

Moreover, the compounds according to the invention can also be used as immune-stimulating or immune modulating active agents.

The present invention also provides pharmaceutical compositions which comprise one or at least one compound according to the invention, optionally in admixture of adjuvants and excipients which are commonly used in the field. The pharmaceutical compositions according to the invention can be formulated/produced according to conventional methods and techniques well-known to one of ordinary skill in the art.

Dosage forms for oral, parenteral (e.g. i.v., s.c., i.p., i.c., intrathecal) and local (e.g. topical, rectal, vaginal, buccal application in the eye or by means of inhalation) application are preferred hereby.

Thus the pharmaceutical composition according to the invention can in particular be presented as tablets (especially also enteric-coated tablets or tablets with a modified release of active agents), capsules (hard and soft gelatine capsules), pills, granulates, suppositories, ovules, ointments, creams, gels, plasters, TTS or also as emulsions, suspensions, solutions or as reconstitutable powders (also for parenteral application).

The embodiments of the invention in which an exclusive property or privilege is claimed are defined as follows:

1. A method for preparing hydroxyacetophenone derivatives of the general formula IV: 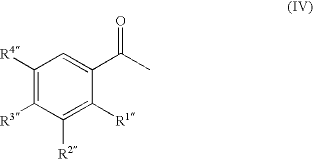
wherein R^{1″}, R^{2″}, R^{3″} and R^{4″} are independently from each other hydrogen, a prenyl group, a 1,1-dimethylallyl group or a hydroxy group, with the proviso that at least one of R^{1″}, R^{2″}, R^{3″} and R^{4″} is a prenyl group or a 1,1-dimethylallyl group and that at least one of R^{1″}, R^{2″}, R^{3″} and R^{4″} is a hydroxy group, comprising reacting a hydroxyacetophenone derivative of the general formula I: 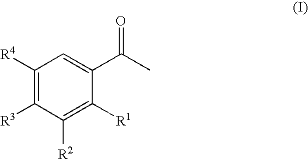
wherein R¹, R², R³ and R⁴ are independently from each other hydrogen, a prenyl group, a 1,1-dimethylallyl group or a hydroxyl group, with the proviso that at least one of R¹, R², R³ and R⁴ is hydrogen and that at least one of R¹, R², R³ and R⁴ is a hydroxy group, in the presence of a base at a reaction temperature of 10 to 50° C., in an organic solvent with a 3-methylbut-2-enylhalide of the formula II: 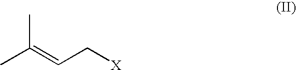
wherein X is chlorine, bromine or iodine, in order to obtain a hydroxyacetophenone derivative of the general formula III: 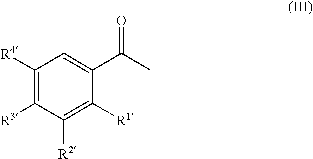
wherein R^{1′}, R^{2′}, R^{3′} and R^{4′} are independently from each other hydrogen, a prenyl group, a 1,1-dimethylallyl group or a 3-methylbut-2-enyloxy group, with the proviso that at least one of R^{1′}, R^{2′}, R^{3′} and R^{4′} is hydrogen and that at least one of R^{1′}, R^{2′}, R^{3′} and R^{4″} is a 3-methylbut-2-enyloxy group, and the reaction of the compound according to formula III in N,N-diethylaniline at a reaction temperature of 160 to 220° C. in order to obtain a hydroxyacetophenone derivative of the general formula IV.

2. The method of claim 1, wherein R^{1′} in the general formula III is a 3-methylbut-2-enyloxy group and R¹ in general formula I is a hydroxyl group.

3. The method of claim 1 or 2, wherein the base is potassium carbonate.

4. The method of claim 1 or 2 which is conducted at a reaction temperature of 20 to 40° C.

5. The method of claim 1 or 2, wherein the organic solvent is dimethylformamide.

6. The method of claim 1, wherein R^{1″} and/or R^{3″} in the general formula IV is a hydroxy group.

7. The method of claim 1 or 6, wherein the reaction of the hydroxyacetophenone derivative of the general formula III to form the hydroxyacetophenone derivative of the general formula IV is conducted under reflux.

8. A method of preparing 4-oxo-4H-chromen-3-carbaldehyde derivatives of the general formula V: 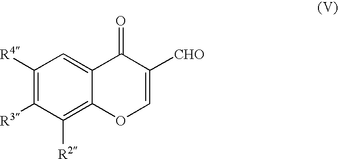
wherein R^{2″}, R^{3″} and R^{4″} are independently from each other hydrogen, a prenyl group, a 1,1-dimethylallyl group or a hydroxy group with the proviso that at least one of R^{2″}, R^{3″} and R⁴″ is a prenyl group or a 1,1-dimethylallyl group, characterised in that a hydroxyacetophenone derivative of general formula IV according to claim 1, wherein R^{1″} is a hydroxy group, is reacted with at least two mole equivalents of dimethylformamide and phosphorus oxychloride at a reaction temperature of 40 to 50° C. in order to obtain the 4-oxo-4H-chromen-3-carbaldehyde derivative of the general formula V.

9. A hydroxyacetophenone derivative of the general formula III: 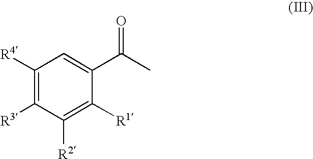
wherein R^{1′}, R^{2′}, R^{3′} and R^{4′} are independently from each other hydrogen, a prenyl group, a 1,1-dimethylallyl group or a 3-methylbut-2-enyloxy group with the proviso that at least one of R^{1′}, R^{2′}, R^{3′} and R^{4′} is hydrogen and that at least one of R^{1′}, R^{2′}, R^{3′} and R^{4′} is a 3-methylbut-2-enyloxy group, wherein 1-[2-(3-methylbut-2-enyloxy)phenyl]ethanone, 1-[4-(3-methylbut-2-enyloxy)phenyl]ethanone and 1-[2,4-bis(3-methylbut-2-enyloxy)phenyl]ethanone are excluded.

10. A hydroxyacetophenone derivative of claim 9, comprising:
1-[5-(3-methylbut-2-enyl)-2-(3-methylbut-2-enyloxy)phenyl]ethanone,
1-[3-(1,1-dimethylallyl)-2-(3-methylbut-2-enyloxy)phenyl]ethanone or
1-[5-(3-methylbut-2-enyl)-4-(3-methylbut-2-enyloxy)phenyl]ethanone.

11. A hydroxyacetophenone derivative of the general formula IV: 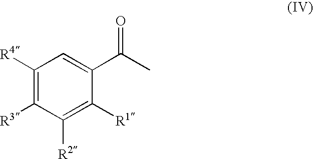
wherein R^{1″}, R^{2″}, R^{3″} and R^{4″} are independently from each other hydrogen, a prenyl group, a 1,1-dimethylallyl group or a hydroxy group with the proviso that at least one of R^{1″}, R^{2″}, R^{3″} and R^{4″} is a prenyl group or a 1,1-dimethylallyl group and that at least one of R^{1″}, R^{2″}, R³″ and R^{4″} is a hydroxy group.

12. A hydroxyacetophenone derivative of claim 11, comprising:
1-[2-hydroxy-5-(3-methylbut-2-enyl)phenyl]ethanone,
1-[4-hydroxy-5-(3-methylbut-2-enyl)phenyl]ethanone,
1-[3-(1,1-dimethylallyl)-2-hydroxyphenyl]ethanone,
1-[3-(1,1-dimethylallyl)-2-hydroxy-5-(3-methylbut-2-enyl)phenyl]ethanone,
1-[2-hydroxy-3,5-bis(3-methylbut-2-enyl)phenyl]ethanone,
1-[4-hydroxy-3,5-bis(3-methylbut-2-enyl)phenyl]ethanone or
1-[2,4-dihydroxy-3,5-bis(3-methylbut-2-enyl)phenyl]ethanone.

13. A 4-oxo-4H-chromen-3-carbaldehyde derivative of the general formula V: 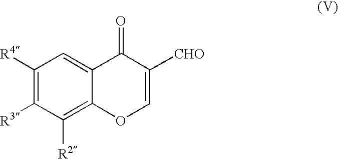
wherein R^{2″}, R^{3″} and R^{4″} are independently from each other hydrogen, a prenyl group, a 1,1-dimethylallyl group or a hydroxy group, with the proviso that at least one of R^{2″}, R^{3″} and R^{4″} is a prenyl group or a 1,1-dimethylallyl group.

14. A 4-oxo-4H-chromen-3-carbaldehyde derivative of claim 13, comprising:
6-(3-methylbut-2-enyl)-4-oxo-4H-chromen-3-carbaldehyde,
6,8-bis(3-methylbut-2-enyl)-7-hydroxy-4-oxo-4H-chromen-3-carbaldehyde,
6,8-bis(3-methylbut-2-enyl)-4-oxo-4H-chromen-3-carbaldehyde,
8-(1,1-dimethylallyl)-6-(3-methylbut-2-enyl)-4-oxo-4H-chromen-3-carbaldehyde or
8-(1,1-dimethylallyl)-4-oxo-4H-chromen-3-carbaldehyde.

15. A pharmaceutical composition comprising at least one compound selected from a compound of claim 9, claim 10, claim 11, claim 12, claim 13, claim 14, 1-[2-(3-methylbut-2-enyloxy)phenyl]ethanone, 1-[4-(3-methylbut-2-enyloxy)phenyl]ethanone and 1-[2,4-bis(3-methylbut-2-enyloxy)phenyl]ethanone.

16. A method of treating a subject in need of such treatment comprising administering to the subject a compound of claim 9, claim 10, claim 11, claim 12, claim 13, claim 14, 1-[2-(3-methylbut-2-enyloxy)phenyl]ethanone, 1-[4-(3-methylbut-2-enyloxy)phenyl]ethanone or 1-[2,4-bis(3-methylbut-2-enyloxy)phenyl]ethanone in an amount effective to achieve an antiproliferative, antimicrobial (for bacteria and fungi), antiviral, immune-modulating or immune-stimulating pharmaceutical effect.

17. The method of claim 16 wherein the compound is administered for the prophylaxis or therapy of neoplastic diseases selected from lung cancer, mamma carcinoma (normal and expressing the MDR phenotype), melanoma, leukaemia, colon cancer, kidney cancer, ovarian cancer, pancreas cancer and prostate cancer.

18. The method of claim 16 wherein the compound is administered for the prophylaxis or therapy of an infection caused by a micro-organism of the genus Candida, Bacillus, Micrococcus, Rhodococcus, Escherichia, Salmonella, Canidida, Enterococcus, Staphylococcus, Neisseria, Salmonella, Pseudomonas, Treponema, Mycobacterium or Trichomonas.

19. The method of claim 18 wherein the compound is administered for the prophylaxis or therapy of an infection, which is caused by a microorganism selected from the group consisting of Bacillus subtilis, Micrococcus luteus, Rhodococcus equi, Enterococcus faecium, Salmonella choterasuis, Escherichia coli, Candida albicans, Staphylococcus aureus and S. epidermides, S. falcium, Neisseria gonorrhoeae, Salmonella pullorum, Pseudomonas aeruginosa, Treponema pallidum, Bacillus coagulans, Mycobacterium leprae, Trichomonas vaginalis, Bacillus cereus, Bacillus megaterium, Micrococcus roseus, Micrococcus varians, Salmonella typhi, Candida albicans or Mycobacterium tuberculosis.
